(19) United States
(12) Patent Application Publication
(10) Pub. No.: US 2009/0287052 A1
(43) Pub. Date: Nov. 19, 2009

(54) Biopsy Cap Attachment and Integrated Locking Device

(75) Inventors: Michael Devon Amos, Boston, MA (US); Jason Weiner, Waltham, MA (US); Mark Wood, Shrewsbury, MA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/467,947

(22) Filed: May 18, 2009

Related U.S. Application Data
(60) Provisional application No. 61/054,294, filed on May 19, 2008.

(51) Int. Cl. A61B 1/00

(52) U.S. Cl. 600/127

(57) ABSTRACT

Endoscope assemblies and methods for making and using the same. An example endoscope assembly may include an endoscope having a channel formed therein and a port that provides access to the channel, a cap disposed on the port, and an attachment assembly coupled to the cap. The attachment assembly may include a base and one or more arms extending from the base. A locking member may be coupled to at least one of the one or more arms.

### CROSS-REFERENCE TO RELATED APPLICATIONS

This application claims the benefit of U.S. Provisional Application No. 61/054,294, filed May 19, 2008, the entire disclosure of which is incorporated herein by reference.

This application is related to U.S. patent application Ser. No. ______ (atty. docket number 1001.2133101), filed on even date herewith and entitled “Integrated Locking Device with Active Sealing”, which claims the benefit of U.S. Provisional Application Ser. No. 61/054,393, filed May 19, 2008; U.S. patent application Ser. No. ______ (atty. docket number 1001.2134101), filed on even date herewith and entitled “Integrated Locking Device with Passive Sealing”, which claims the benefit of U.S. Provisional Application Ser. No. 61/054,407, filed May 19, 2008; and U.S. patent application Ser. No. ______ (atty. docket number 1001.2135101), entitled “Integrated Locking Device with Fluid Control”, filed on even date herewith, which claims the benefit of U.S. Provisional Application No. 61/054,413, filed May 19, 2008; which disclosures are all hereby incorporated herein by reference. This application is also related to U.S. patent application Ser. No. 12/029,148, filed Feb. 11, 2008, the disclosure of which is incorporated herein by reference.

### FIELD OF THE INVENTION

The present invention pertains to endoscopes and medical devices for use with endoscopes. More particularly, the present invention pertains to caps for an access port of an endoscope and attachments and/or locking devices disposed adjacent to the caps.

### BACKGROUND

A wide variety of endoscopes and/or endoscope assemblies, medical devices for use with endoscopes, and endoscopic procedures have been developed. Of the known endoscopes, medical devices for use with endoscopes, and endoscopic procedures, each has certain advantages and disadvantages. There is an ongoing need to provide alternative endoscopes as well as methods for making and using endoscopes.

### BRIEF SUMMARY

The invention provides design, material, and manufacturing method alternatives for endoscopes, medical devices for use with endoscopes and/or endoscope assemblies, caps for an access port of an endoscope, attachments and/or locking devices disposed adjacent to the caps, and methods for making and using endoscopes. An example endoscope assembly may include an endoscope having a channel formed therein and a port that provides access to the channel, a cap disposed on the port, and an attachment assembly coupled to the cap. The attachment assembly may include a base and one or more arms extending from the base. A locking member may be coupled to at least one of the one or more arms.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of an example endoscope assembly;

FIG. 2 is an exploded view of a portion of the example endoscope assembly shown in FIG. 1 illustrating a biopsy cap and an attachment member;

FIG. 3 is a side view illustrating an example attachment member coupled to a biopsy cap;

FIG. 4 illustrates an example locking member for an attachment member;

FIG. 5 illustrates another example locking member for an attachment member;

FIG. 6 illustrates another example locking member for an attachment member;

FIG. 6A illustrates an alternative configuration for the example locking member illustrated in FIG. 6;

FIG. 7 illustrates another example locking member for an attachment member;

FIG. 8 illustrates another example locking member for an attachment member; and

FIG. 9 illustrates another example locking member.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

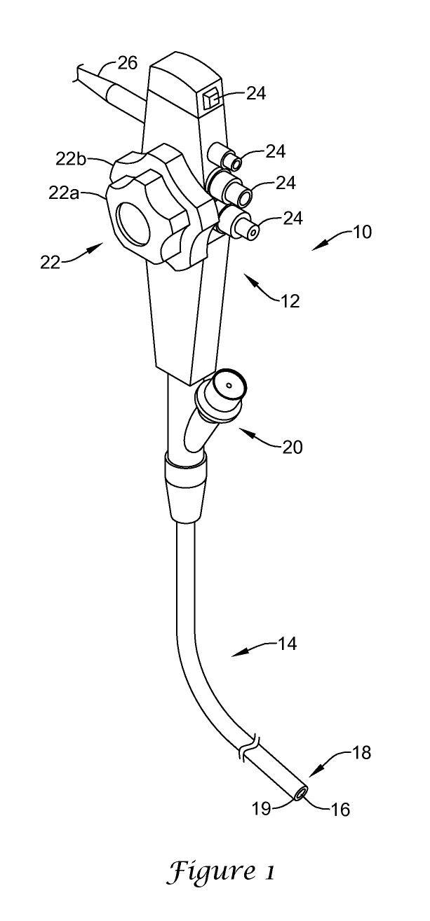

An example endoscope and/or endoscope assembly 10 is illustrated in FIG. 1. Endoscope 10 may be any of a number of types of endoscopes or related medical devices usually identified by the particular anatomy desired to be reached. For example, endoscope 10 may be a bronchoscope, colonoscope, duodenoscope, esophagoscope, or any other type of endoscope or related medical device. Endoscope 10 may include a handpiece 12 and an elongate shaft 14 extending distally from handpiece 12 to a distal tip 18. Shaft 14 may include a lumen defining a working channel 16 extending through shaft 14 from a distal end 19 near distal tip 18 of shaft 14 to an access port 20 that may be positioned in handpiece 12 or another portion of endoscope 10. Although endoscope 10 is depicted with a single working channel in FIG. 1, it can be appreciated that in other embodiments, endoscope 10 may include multiple working channels, as desired.

Handpiece 12 may include one or a plurality of controls 22, such as rotating knobs, which may be used to control movement of distal end 18 of shaft 14 during operation. For example, a first rotating knob 22a may control up and down movement or deflection of distal tip 18 of shaft 14, while a second rotating knob 22b may control side-to-side movement or deflection of distal tip 18 of shaft 14. Handpiece 12 may also include one or a plurality of buttons 24, which may be used to activate suction or deliver fluid such as air, saline and/or water, etc. through a lumen of the endoscope 10 or perform other functions as desired. Additionally, handpiece 12 may include an optical Is cable 26 connected to an external light source (not shown).

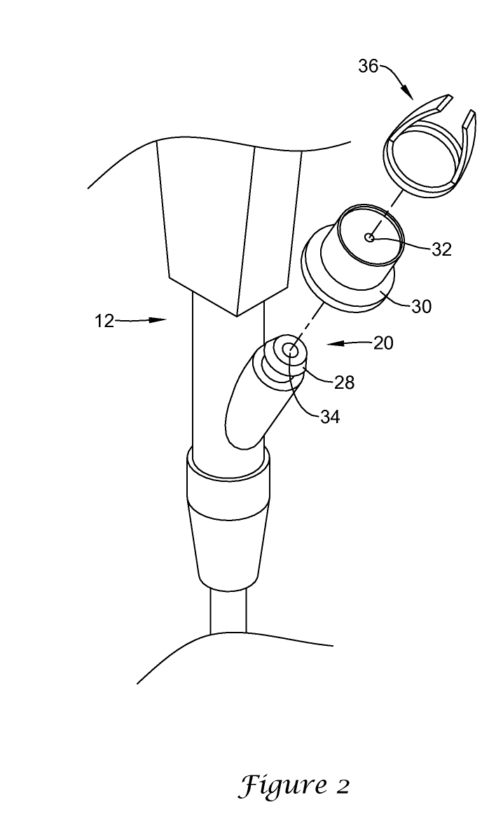

Turning now to FIG. 2, here access port 20 of handpiece 12, which provides access to working channel 16 of endoscope 10, is illustrated. Access port 20, which may extend from the side of endoscope 10 or at another location, may include a coupling portion 28 for coupling a cap 30 to access port 20. Cap 30, which may be removably attached or permanently attached to access port 20, may provide access for inserting and/or advancing an endoscopic device through working channel 16 of endoscope 10.

Caps like cap 30, which may be termed “biopsy caps”, are often designed with several functions in mind. For example, cap 30 may form a fluid/air barrier to working channel 16 that may help control insufflation and bile fluid egress therefrom, that later have the potential to spill onto the clinician's hands and/or the floor thereby interfering with the intervention and/or become a biohazard. In addition, cap 30 may have an opening 32 extending therethrough. Opening 32 may be in fluid communication with working channel 16 and it may reduce the size of the opening 34 of working channel 16, for example, to accommodate an endoscopic device or instrument. Thus, caps like cap 30 may be much like an adapter in that it forms a physical transition at opening 34 of working channel 16 so that it transitions to a size more close to that of the device to be inserted into working channel 16.

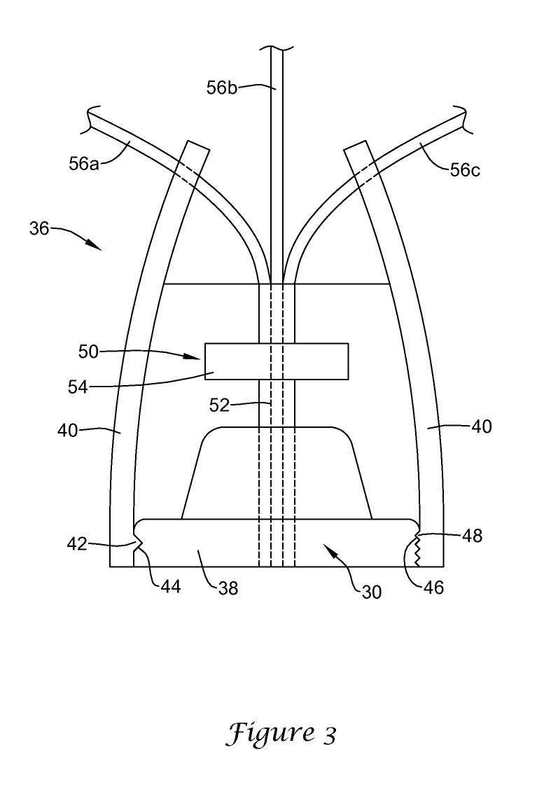

Biopsy caps, for example like cap 30, lack a structure for locking the position (e.g., relative to endoscope 10) of a guidewire and/or another device (e.g., catheter, sphincterotome, basket, biopsy forceps, snare, combinations thereof, or the like) extending through opening 32. To fulfill this need, endoscope 10 may include an attachment member 36 that may be secured to cap 30 and/or port 20 that includes one or more structures for securing the position of a guidewire and/or device used in conjunction with endoscope 10. Attachment member 36 may include a base 38 and one or more arms 40 extending from base 38 as illustrated in FIG. 3. Attachment member 36 may be constructed from generally rigid materials (some examples of suitable materials are listed below) so that attachment member 36 can maintain its integrity while performing its guidewire and/or device locking functions. Of course, any suitable material may be utilized.

Base 38 may take the form of a generally round disk that is configured to be disposed about cap 30. In at least some embodiments, base 38 goes all the way around cap 30. In other embodiments, base 38 may be discontinuous so that it travels only part of the way around cap 30.

In general, base 38 of attachment 36 may be releasably attachable to cap 30 (and/or port 20). The precise manner in which attachment 36 is secured to cap 30 may vary considerably. For example, base 38 may include a protrusion or ridge 42 along the interior of base 38 that may mate with a valley or groove 44 on cap 30. According to this embodiment, attachment 36 may be fitted over cap 30 such that ridge 42 engages groove 44, thereby securing and holding attachment 36 on cap 30. Alternatively, base 38 may include a thread 46 that may similarly mate with a complementary thread 48 formed on cap 30. It is worth noting that both the ridge/groove version and the complementary threads version of base 38/cap 30 are depicted in FIG. 3. This, however, is for illustration purposes as these embodiments will typically be used separately from one another on different versions of base 38 and cap 30. Any number of alternative connection mechanisms may be utilized to secure attachment 36 to cap 30 including versions where base 38 fits snugly over cap 30 and is held in place by friction.

In some embodiments, a bile control valve 50 may extend into cap 30, for example through opening 32. Valve 50 may include a first or vertical component 52 that extends through opening 32 and a second or horizontal component 54. Vertical component 52 may include one or more openings therein that allow one or more devices and/or guidewires (e.g., devices and/or guidewires 56a/56b/56c as depicted in FIG. 3) to extend therethrough and into working channel 16. Horizontal component 54 may also include one or more openings and it may function by diverting bile or other fluids that may seep or flow from cap 30 to a suitable collection apparatus. Alternatively, or additionally, horizontal component 54 may be rotatable relative to vertical component 52 such that horizontal component 54 can be rotated to close (i.e., seal) and open the openings in vertical component. Thus, in these embodiments, horizontal component 54 may function as the trigger for valve 50 that seals off cap 30 (e.g., opening 32 of cap 30), for example when devices 56a/56b/56c are absent from valve 50.

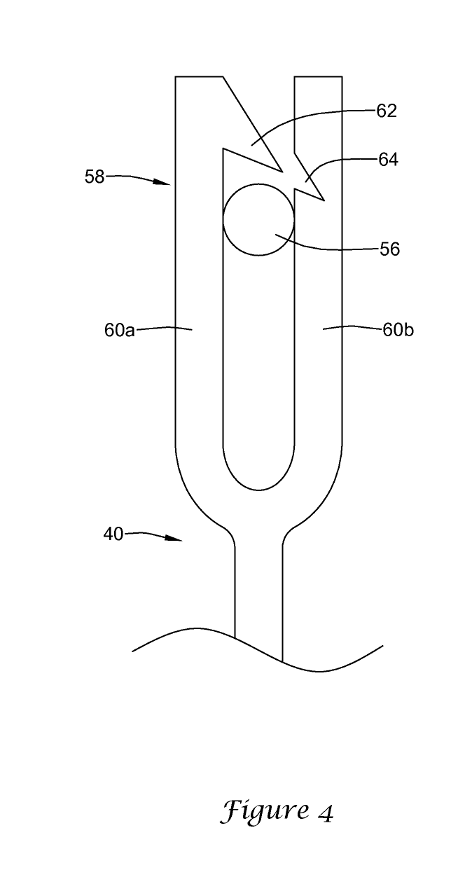

Extending from base 38 are one or more arms 40. The precise number of arms 40 can vary. For example, attachment member 36 may include one, two, three, four, five, six, or more arms 40. Arms 40 may include a locking member or device 58. A number of different embodiments of locking devices are depicted in FIGS. 4-7 that may be used with arms 40. In some embodiments, all of the arms 40 of a given attachment member 36 have the same form of locking member 58. In other embodiments, some of the arms 40 may have different locking members 58. In the embodiment depicted in FIG. 4, locking member 58 may take the form of a pinch clamp. Pinch clamp 58 may include a pair of opposing arms or members 60a/60b. Member 60a may include a notch or protrusion 62 whereas member 60b may include a valley or channel 64. With guidewire or device 56 disposed between members 60a/60b, members 60a/60b may be “pinched” or otherwise brought together so that notch 62 becomes disposed in channel 64. When in this configuration, the space between members 60a/60b is reduced and device 56 is secured therebetween.

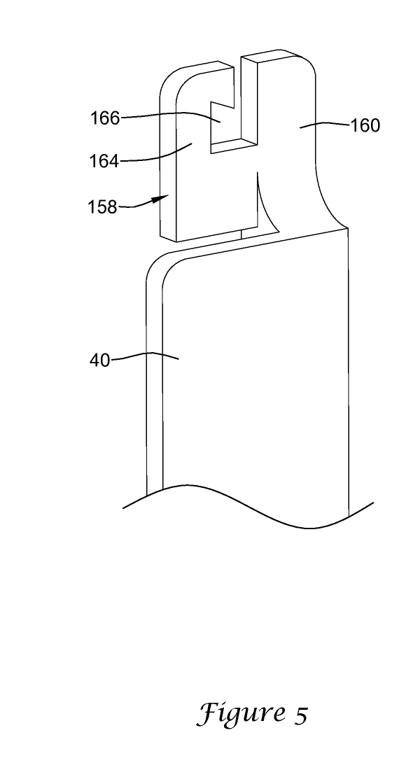

FIG. 5 illustrates another example locking member 158. Locking member 158 may include a tab 160, a hook or locking arm 164 extending from tab 60, and a device opening 166 disposed between tab 160 and arm 164. In use, a device may be extended through opening 166 and “catch” on arm 164. This may effectively hold the position of the device. Alternatively, the device may be wound or wrapped around tab 160 and/or arm 164 to secure the position of the device.

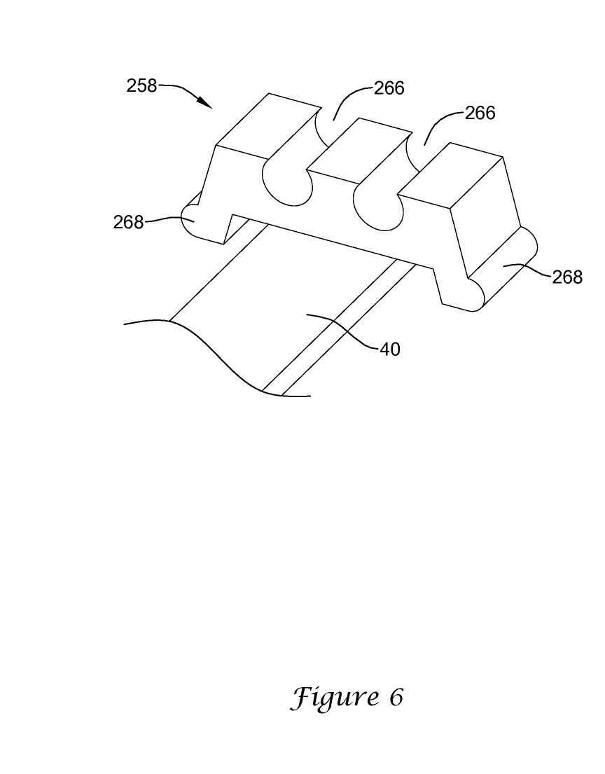

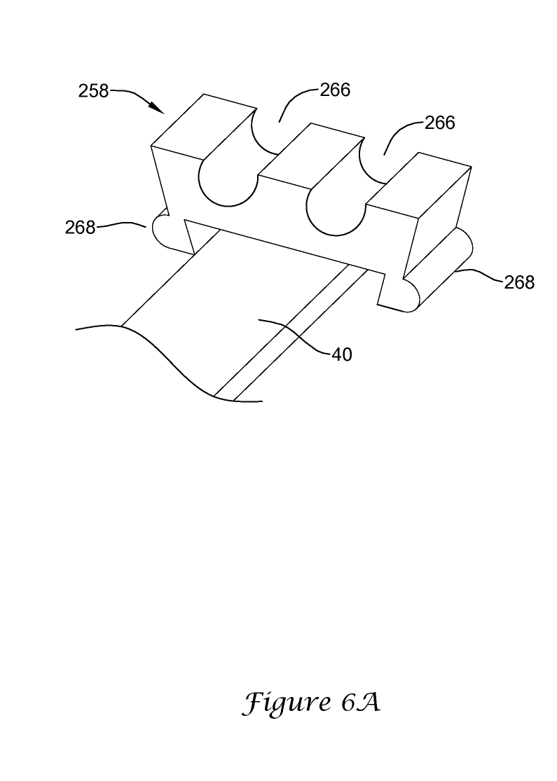

FIG. 6 illustrates another example locking member 258. Locking member 258 may take the form of a clamp that includes one or more channels 266 flanked by a pair of opposing pinch members 268. Locking member 258 may be configured to shift between a first configuration and a second configuration. For example, a clinician may use their fingers to apply pressure to pinch members 268 (e.g., the clinician may pinch together pinch members 268) to widen or “open” channels 266 as depicted in FIG. 6A. While open, a device (e.g., guidewire or device 56) may be disposed in one of channels 266. Once properly positioned, the clinician may remove the pressure that was applied to pinch members 268 so that channels 266 take their original form and “close” around the device, thereby securing the position of the device disposed therein.

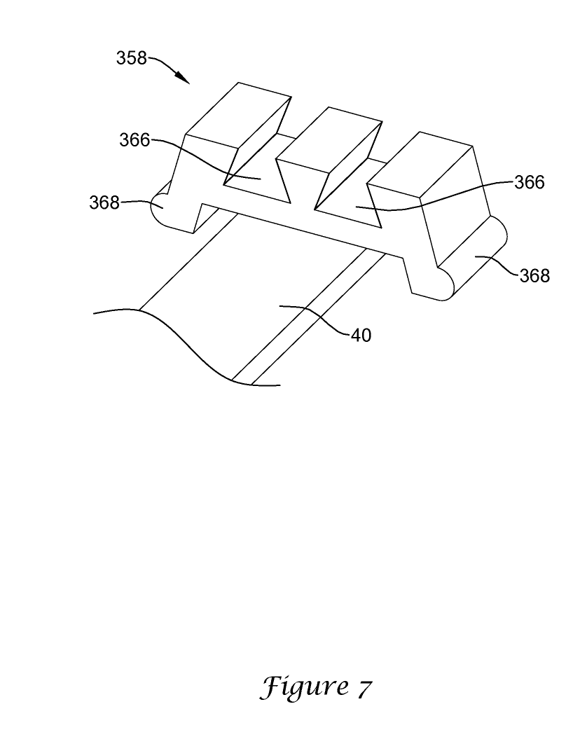

Although channel 266 is shown in FIG. 6 as have a generally rounded or semicircular shape, this is not intended to be limiting. Numerous other shapes are contemplated. For example, FIG. 7 illustrates locking member 358 that takes the form of a clamp with pyramidal channels 366. Much like channels 266, channels 366 can be widened or opened by pinching pinch members 368.

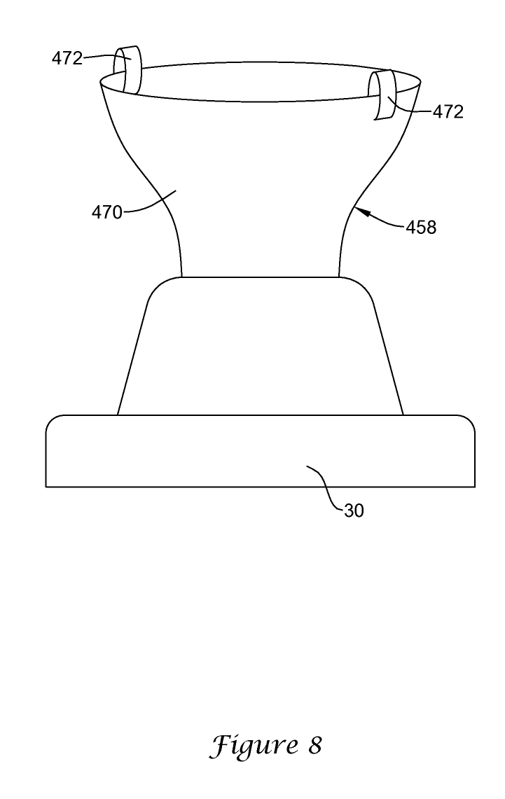

Another example locking member 458 is illustrated in FIG. 8. Locking member 458 may include a funnel-shaped member 470 that may be disposed in opening 32 of cap 30. In some embodiments, funnel-shaped member 470 may be defined by a plurality of arms similar to arms 40 linked by a sheath or linking structure. Alternatively, funnel-shaped member 470 may comprise a unitary conical structure. One or more removable locks 472 may be releasably attachable to funnel-shaped member 470. The form of locks 472 may vary considerably. For example, in some embodiments, locks 472 may take the form of a pinching member that locks the device onto funnel-shaped member 470. The pinching member embodiment of locks 472 may function similarly to a clothes pin. Alternatively, lock 472 may resemble and function similarly to a paper clip. It can be appreciated that a vast number of different types of locks 472 are contemplated and may be used without departing from the spirit of the invention.

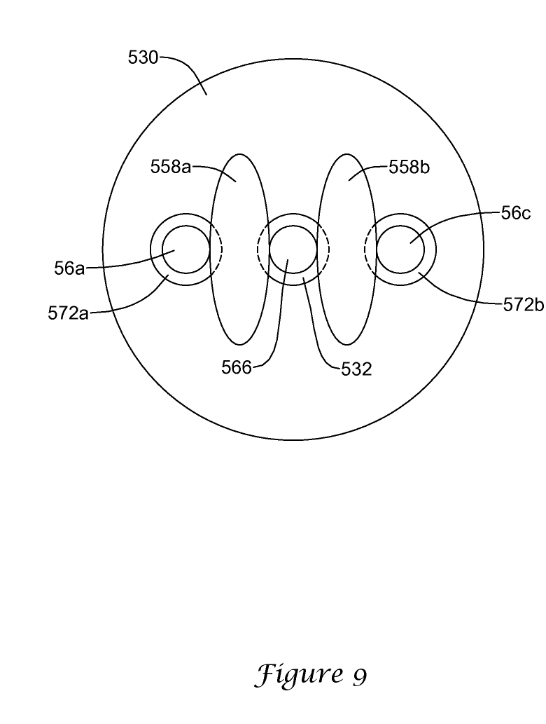

FIG. 9 illustrates another example cap 530 that may be similar in form and function to cap 30. Cap 530 may include a plurality of openings including, for example, opening 532 and guidewire and/or device openings 572a/572b. A guidewire or device (e.g., guidewire or device 56a/56b/56c) may be disposed in any or all of openings 532/572a/572b. One or more locking wedges, for example wedges 558a/558b, may be disposed along cap 530. Wedges 558a/558b may wedge between a pair of adjacent devices (e.g., between device 56a and 56b or between device 56b and 56c) to effectively hold and secure the position of device(s) 56a/56b/56c.

In addition to the locking features or members illustrated above, it can be appreciated that several other alternative locking members may be used to secure the position of a device. Some example of these other locking structures may include a spring clip, a wedge, a plurality of hooks, a spring button, arms that shift between an open and a closed configuration (e.g., like a clothespin), a plurality of fingers that shift between an open and a closed configuration, a base in combination with a platform having a hook-like extension, arms attached by a linkage, locks in combination with a wing or sheath that guides a device toward the lock, and the like, or combinations thereof. Similarly, a number of additional attachment techniques may be used to secure the various attachment members to caps including one or more locking tabs, a plug or stopper configuration, a plurality of fingers, a deformable end or opening, a threaded connection, a quick release pull tab, a slidable button, and the like, or combinations thereof.

The various caps, attachment members, and locking members, as well as the various components thereof may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In at least some embodiments, portions or all of the structures disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of endoscope 10 in determining its location. Some examples of radiopaque materials can include, but are not limited to, gold, platinum, palladium, tantalum, tungsten alloy, polymer material loaded with a radiopaque filler, and the like. Additionally, radiopaque marker bands and/or coils may be incorporated into the design of endoscope 10 or the various components thereof to achieve the same result.

In some embodiments, a degree of MRI compatibility may be imparted into the structures disclosed herein. For example, to enhance compatibility with Magnetic Resonance Imaging (MRI) machines, it may be desirable to make a portion of endoscope 10 in a manner that would impart a degree of MRI compatibility. For example, a portion of endoscope 10 may be made of a material that does not substantially distort the image and create substantial artifacts (artifacts are gaps in the image). Certain ferromagnetic materials, for example, may not be suitable because they may create artifacts in an MRI image. A portion of endoscope 10 may also be made from a material that the MRI machine can image. Some materials that exhibit these characteristics include, for example, tungsten, cobalt-chromium-molybdenum alloys (e.g., NS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An endoscope assembly, comprising:
an endoscope having a channel formed therein and a port that provides access to the channel;
a cap disposed on the port;
an attachment assembly coupled to the cap, the attachment assembly including a base and a plurality of arms extending from the base; and
a locking member coupled to at least one of the plurality of arms.

2. The assembly of claim 1, wherein the locking member includes a pinch clamp.

3. The assembly of claim 1, wherein the locking member includes a hook.

4. The assembly of claim 1, wherein the locking member includes a clamp which is adapted to shift between a first configuration and a second configuration.

5. The assembly of claim 4, wherein the clamp has one or more channels formed therein, and wherein the channels are substantially open when the clamp is in the first configuration and the channels are substantially closed when the clamp is in the second configuration.

6. The assembly of claim 1, wherein the cap includes an opening and wherein the one or more arms define a funnel member disposed in the opening.

7. The assembly of claim 6, wherein the locking member is detachably connected to the funnel member.

8. An endoscope biopsy cap attachment, comprising:
a round base having an end surface configured to be disposed about and attach to an endoscope biopsy cap; and
one or more locking members disposed adjacent to the base, the locking members being configured to secure an endoscopic instrument, a guidewire, or both during an endoscopic medical procedure.

9. The endoscope biopsy cap attachment of claim 8, wherein an arm extends from the base and wherein at least one of the one or more locking members are disposed on the arm.

10. The endoscope biopsy cap attachment of claim 9, wherein the locking member includes a pinch clamp.

11. The endoscope biopsy cap attachment of claim 9, wherein the locking member includes a hook.

12. The endoscope biopsy cap attachment of claim 9, wherein the locking member includes clamp.

13. The endoscope biopsy cap attachment of claim 8, wherein the locking member includes a wedge member.

14. The endoscope biopsy cap attachment of claim 8, wherein the locking member includes a funnel having one or more locks detachably connected to the funnel member.

15. The endoscope biopsy cap attachment of claim 8, wherein the end surface of the base includes a ridge.

16. The endoscope biopsy cap attachment of claim 8, wherein the end surface of the base includes a thread.

17. An endoscope assembly, comprising:
an endoscope having a channel formed therein and a port that provides access to the channel;
a cap disposed on the port;
a medical device extending through the port and into the channel; and
means for securing the position of the medical device relative to the cap, the means for securing the position of the medical device relative to the cap being disposed adjacent to the cap.

18. The endoscope assembly of claim 17, wherein the medical device is an endoscopic instrument.

19. The endoscope assembly of claim 17, wherein the medical device is a guidewire.

20. A method of medical treatment, the method comprising:
providing an endoscope having a channel formed therein and a port that provides access to the channel;
disposing the endoscope within a body lumen of a patient;
disposing a cap on the port; and
coupling an attachment assembly cap, the attachment assembly including a base, one or more arms extending from the base, and a locking member coupled to at least one of the one or more arms.
